(19) United States
(12) Patent Application Publication
(10) Pub. No.: US 2022/0015667 A1
(43) Pub. Date: Jan. 20, 2022

(54) SYSTEMS AND METHODS FOR OBTAINING AND MONITORING RESPIRATION, CARDIAC FUNCTION, AND OTHER HEALTH DATA FROM PHYSICAL INPUT

(71) Applicants: Samantha Kurkowski, Maryland Heights, MO (US); George Chronis, Chesterfield, MO (US)

(72) Inventors: Samantha Kurkowski, Maryland Heights, MO (US); George Chronis, Chesterfield, MO (US)

(21) Appl. No.: 17/381,002

(22) Filed: Jul. 20, 2021

Related U.S. Application Data
(63) Continuation-in-part of application No. 17/228,249, filed on Apr. 12, 2021.
(60) Provisional application No. 63/054,078, filed on Jul. 20, 2020.

(51) Int. Cl. A61B 5/08; A61B 5/00; A61B 5/024

(52) U.S. Cl. CPC A61B 5/0816; A61B 5/6892; A61B 2562/0271; A61B 5/0823; A61B 2562/0219; A61B 5/024

(57) ABSTRACT

A sensor which is generally included as part of a bed or chair or is attached to or placed on or under a mattress for the physical detection of respiration and cardiac function and particularly alteration in respiration or cardiac function. The sensor may be a portion of a system which is designed to monitor respiration and cardiac function over time.

### CROSS REFERENCE TO RELATED APPLICATION(S)

This application claims the benefit of U.S. Provisional Patent Application Ser. No. 63/054,078, filed Jul. 20, 2020. This application is also a Continuation-In-Part (CIP) of U.S. Utility patent application Ser. No. 17/228,249 filed Apr. 12, 2021. The entire disclosure of all the above documents is herein incorporated by reference.

### BACKGROUND OF THE INVENTION

### Field of the Invention

The present disclosure relates to physical sensors for the detection of specific health related conditions and systems and methods which can utilize that output. In particular, to physical sensors that monitor physical body changes in respiration and heart rate.

### Description of the Related Art

Motion sensors and detectors are already in use in a variety of medical areas. For example, sensors located in a patient's bed or on a chair can be used to sense when a patient has gotten up, is attempting to stand, or has not moved for a period of time. These kinds of sensors can be used to detect that a patient is currently at an increased risk of falling, issue warnings before possible falls occur, detect falls when they occur, and alert against pressure ulcers in both hospitals and senior care facilities.

In the wake of the 2020 COVID-19 virus pandemic, the human population saw the global spread of a deadly disease leading to mass “social distancing” and the use of respiratory masks in an attempt to halt its spread before vaccines could be made available. Social distancing was effectively a lighter form of quarantine where all individuals were intended simply to be kept at a distance from each other so that COVID-19, which was believed to be spread from an infected individual to others primarily by airborne transmission, did not pass from an infected person (who may not have been aware they were infected) to one that was not. Masks or other face coverings, which ranged from homemade sewn fabric constructions to advanced N95 particulate respirators, were also widely used to attempt to block airborne particulates ejected by one person from being inhaled by another.

The effectiveness of these measures is the subject of some debate and because of the concern as to the various measures' effectiveness, a number of other tests were commonly used to attempt to determine if someone was sick, or more importantly, contagious. For example, guidelines for interacting with others during the pandemic often required or suggested that an individual would need to have a normal (e.g. not elevated) body temperature to attend group events. For example, individuals such as children attending school or camp or adults entering workplaces often had to have their temperature taken, or to assert that they had taken it and it was normal, before entering and being allowed to interact with others.

The purpose of all this temperature taking was that elevated body temperature is an indicator that the body is fighting infection. Thus, if one has elevated body temperature, the likelihood that they have some form of infection is increased. However, all this temperature taking was also still being done even when evidence began to show that temperature was likely a lagging indicator of COVID-19 infection as temperature usually is. This became relevant because the individual was believed to possibly be contagious long before they had elevated body temperature.

As temperature taking during the COVID-19 pandemic illustrated, one problem with many commonly used indications of sickness or injury is that they are lagging indicators. Often, the indicators are only interrogated when a person “feels” sick which may mean that the indicator is good at confirming illness, but not good at actually detecting it.

The risk of another pandemic should not be trifled with. However, often with infectious disease the virus or pathogen itself is not the danger, but the human body's response to the virus or pathogen is. The body's response often results in problems that are what actually causes permanent injury or death to the individual. For example, for respiratory ailments, respiratory distress including the generation of excess mucus in the respiratory system along with inflammation which can lead to coughing, nasal symptoms such as congestion (rhinitis) and runny nose (rhinorrhea), headaches, and general weakness from the body's reduced capacity to handle air. In other diseases, the virus response can result in damage to blood cells which can in turn result in damage to blood vessels and heart tissue.

Outside of infection, degenerative illness, acute conditions, and simple aging can also cause changes to the operation of the body which can result in both acute dangers or long term degeneration in similar fashion to acute illness. For example, heart attacks result in the heart stopping which can result in death if not detected quickly. While heart attacks are an acute condition with immediate concern, the causes of heart attacks are often degenerative and take many months if not years of accumulation before the danger is realized. Similarly, degenerative illnesses such as Amyotrophic Lateral Sclerosis (ALS) can cause loss of motor control which can result in trouble breathing due to inability to accurately control the lungs.

Detection of the alteration of the function of major body systems can provide an indication of potential disease, degeneration, or other condition in a way that allows for it to be corrected, before it becomes a problem. For example, screenings for various forms of cancer are common as they often allow for detection of the presence of a tumor before it has grown and spread in a way that is not easily removed. Some of the most major body functions for life revolve around cardiac function and respiration. However, disease in these areas if often degenerative and by the time a problem is identified, it often requires radical measures to correct. For example, it is fairly well recognized that clogged arteries causes the heart to work harder and can result in heart failure. However, it is often hard to detect that the heart is working harder than before because heart monitoring can be difficult to perform, invasive, and may require circumstances which are not everyday occurrences.

### SUMMARY OF THE INVENTION

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, described herein are sensors which are generally included as part of a bed or chair or is attached to or placed on or under a mattress (but may be otherwise positioned to detect movement of a human torso) for the physical detection of respiration and cardiac function and particularly alteration in respiration or cardiac function. The sensor may be a portion of a system which is designed to monitor respiration and cardiac function over time.

There is described herein, among other things, a sensor for detecting cardiac function or breathing of an animal, the sensor comprising: a support formed of a base and an outer wall; an accelerometer; and a spring, said spring at equilibrium suspending said accelerometer above said base; wherein when said spring allows for said accelerometer to be moved relative to said base.

In an embodiment of the sensor, the animal is a human.

In various embodiments of the sensor, the spring comprises elastic bands, an elastic flat surface material, flat spring steel, a leaf spring, a coil spring, and/or a disk spring.

In an embodiment of the sensor, the spring is attached to said outer wall.

In an embodiment of the sensor, there is a void between said spring and said base which said accelerometer can move into.

There is also described herein a system for detecting cardiac function or breathing of an animal, the system comprising: a sensor comprising: a support formed of a base and an outer wall; an accelerometer; and a spring, said spring at equilibrium suspending said accelerometer above said base; wherein when said spring allows for said accelerometer to be moved relative to said base; and a support structure, mounting said sensor in a fashion that movement of a human torso through respiration or cardiac function causes said accelerometer to move relative to said base.

In an embodiment of the system, the support structure comprises a bed.

In an embodiment of the system, the support structure comprises a chair.

In an embodiment of the system, the support structure comprises said human torso.

In an embodiment, the system further comprises a central computer for receiving signals from said accelerometer via a network.

In an embodiment, the system further comprises a thermal sensor.

In an embodiment, the system further comprises a motion sensor.

In an embodiment, the system further comprises a depth sensor.

In an embodiment of the system, the central computer can compare said received signals against other signals.

In an embodiment of the system, the other signals comprise prior received signals.

### BRIEF DESCRIPTION OF THE DRAWINGS

FIG. 1 depicts a general overview of an embodiment of a composite system for obtaining and monitoring respiration, heart rate, and other health data from physical input utilising a physical sensor.

FIG. 2A shows a first embodiment of a sensor primarily for detecting motion of a patient's chest and which is suitable for inclusion within, on, or under a mattress or chair cushion or may be placed on a patient directly.

FIG. 2B shows a second embodiment of a sensor primarily for detecting motion of a patient's chest and which is suitable for inclusion within, on, or under a mattress or chair cushion or may be placed on a patient directly.

FIG. 3 shows a third embodiment of a sensor for primarily detecting motion of a patient's chest and which is suitable for inclusion within, on or under a mattress or chair cushion or may be placed on a patient directly.

FIG. 4 shows a graph of output from a motion-detecting sensor showing detection of a patient's heartbeat.

FIG. 5 shows two graphs of a motion-detecting sensor showing detection of the same patient as FIG. 4's respiration pattern.

FIG. 6 shows two graphs of a motion-detecting sensor showing detection of the same patient as FIG. 5's respiration pattern but using a different detected acceleration methodology.

FIG. 7 shows two graphs of a motion-detecting sensor showing detection of the respiration pattern of two different patients from the patient of FIG. 4, but using the methodology of FIG. 5.

FIG. 8 shows six graphs of a motion-detecting sensor detecting coughs and periods of fast breathing in six different patients.

### DETAILED DESCRIPTION OF PREFERRED EMBODIMENTS

Throughout this disclosure, the term “physical detection” or “mechanical detection” is used to broadly refer to technologies which detect changes in the physical world as opposed to chemical detection or biological detection. Physical detection in this disclosure will commonly utilize changes in movement (including starting, stopping, direction, acceleration, or velocity), or changes in orientation. Physical detection also includes changes to electrical or magnetic fields as well as alteration of structural processes and subatomic forces. In many respects, physical detection relates to detection of any change within electromechanical parameters in the operation of the human body as opposed to biological or chemical changes (although such biological and chemical changes are recognized as often causing the changes in electromechanical parameters). Physical detection can be carried out by a wide variety of instruments, but devices such as motion detectors (including cameras in all electromagnetic spectrums), accelerometers, magnetometers, gyroscopes, and other types of well-known measurement devices are all capable of physical detection. Physical parameters including, but not limited to, acceleration, g-force, angular velocity or change in magnetic field can all be measured.

The term “computer” describes hardware which generally implements functionality provided by digital computing technology, particularly computing functionality associated with microprocessors. The term “computer” is not intended to be limited to any specific type of computing device, but it is intended to be inclusive of all computational devices including, but not limited to: processing devices, microprocessors, personal computers, desktop computers, laptop computers, workstations, terminals, servers, clients, portable computers, handheld computers, cell phones, mobile phones, smart phones, tablet computers, server farms, hardware appliances, minicomputers, mainframe computers, video game consoles, handheld video game products, and wearable computing devices including, but not limited to eyewear, wristwear, pendants, fabrics, and clip-on devices.

As used herein, a “computer” is necessarily an abstraction of the functionality provided by a single computer device outfitted with the hardware and accessories typical of computers in a particular role. By way of example and not limitation, the term “computer” in reference to a laptop computer would be understood by one of ordinary skill in the art to include the functionality provided by pointer-based input devices, such as a mouse or track pad, whereas the term “computer” used in reference to an enterprise-class server would be understood by one of ordinary skill in the art to include the functionality provided by redundant systems, such as RAID drives and dual power supplies.

It is also well known to those of ordinary skill in the art that the functionality of a single computer may be distributed across a number of individual machines. This distribution may be functional, as where specific machines perform specific tasks; or, balanced, as where each machine is capable of performing most or all functions of any other machine and is assigned tasks based on its available resources at a point in time. Thus, the term “computer” as used herein, can refer to a single, standalone, self-contained device or to a plurality of machines working together or independently, including without limitation: a network server farm, “cloud” computing system, software-as-a-service (SAAS), or other distributed or collaborative computer networks.

Those of ordinary skill in the art also appreciate that some devices which are not conventionally thought of as “computers,” nevertheless exhibit the characteristics of a “computer” in certain contexts. Where such a device is performing the functions of a “computer” as described herein, the term “computer” includes such devices to that extent. Devices of this type include, but are not limited to: network hardware, print servers, file servers, NAS and SAN, load balancers, and any other hardware capable of interacting with the systems and methods described herein in the matter of a conventional “computer.”

Throughout this disclosure, the term “software” refers to code objects, program logic, command structures, data structures and definitions, source code, executable and/or binary files, machine code, object code, compiled libraries, implementations, algorithms, libraries, or any instruction or set of instructions capable of being executed by a computer processor, or capable of being converted into a form capable of being executed by a computer processor, including, without limitation, virtual processors, or by the use of run-time environments, virtual machines, and/or interpreters. Those of ordinary skill in the art recognize that software can be wired or embedded into hardware, including, without limitation, onto a microchip, and still be considered “software” within the meaning of this disclosure. For purposes of this disclosure, software includes, without limitation: instructions stored or storable in hard drives, RAM, ROM, flash memory BIOS, CMOS, mother and daughter board circuitry, hardware controllers, USB controllers or hosts, peripheral devices and controllers, video cards, audio controllers, network cards, Bluetooth® and other wireless communication devices, virtual memory, storage devices and associated controllers, firmware, and device drivers. The systems and methods described here are contemplated to use computers and computer software typically stored in a computer- or machine-readable storage medium or memory.

Throughout this disclosure, the term “network” generally refers to a voice, data, or other telecommunications network over which computers communicate with each other. The term “server” generally refers to a computer providing a service over a network, and a “client” generally refers to a computer accessing or using a service provided by a server over a network. Those having ordinary skill in the art will appreciate that the terms “server” and “client” may refer to hardware, software, and/or a combination of hardware and software, depending on context. Those having ordinary skill in the art will further appreciate that the terms “server” and “client” may refer to endpoints of a network communication or network connection, including, but not necessarily limited to, a network socket connection. Those having ordinary skill in the art will further appreciate that a “server” may comprise a plurality of software and/or hardware servers delivering a service or set of services. Those having ordinary skill in the art will further appreciate that the term “host” may, in noun form, refer to an endpoint of a network communication or network (e.g., “a remote host”), or may, in verb form, refer to a server providing a service over a network (“hosts a website”), or an access point for a service over a network.

Throughout this disclosure, the term “transmitter” refers to equipment, or a set of equipment, having the hardware, circuitry, and/or software to generate and transmit electromagnetic waves carrying messages, signals, data, or other information. A transmitter may also comprise the componentry to receive electric signals containing such messages, signals, data, or other information, and convert them to such electromagnetic waves. The term “receiver” refers to equipment, or a set of equipment, having the hardware, circuitry, and/or software to receive such transmitted electromagnetic waves and convert them into signals, usually electrical; from which the message, signal, data, or other information may be extracted. The term “transceiver” generally refers to a device or system that comprises both a transmitter and receiver, such as, but not necessarily limited to, a two-way radio, or wireless networking router or access point. For purposes of this disclosure, all three tell is should be understood as interchangeable unless otherwise indicated; for example, the term “transmitter” should be understood to imply the presence of a receiver, and the term “receiver” should be understood to imply the presence of a transmitter.

For purposes of this disclosure, there will also be significant discussion of a special type of computer referred to as a “mobile communication device” or simply “mobile device”. A mobile communication device may be, but is not limited to, a smart phone, tablet PC, e-reader, satellite navigation system (“SatNav”), fitness device (e.g. a Fitbit™ or Jawbone™) or any other type of mobile computer, whether of general or specific purpose functionality. Generally speaking, a mobile communication device is network-enabled and communicating with a server system providing services over a telecommunication or other infrastructure network. A mobile communication device is essentially a mobile computer, but one which is commonly not associated with any particular location, is also commonly carried on a user's person, and usually is in near-constant real-time communication with a network.

The system may utilize a “positioning system” which is any form of location technology and will typically be a satellite positioning system such as GPS, GLONASS, or similar technology, but may also include inertial and other positioning systems, and wireless communication to enable detection of location such as beacon technology. Any wireless methodology for transferring the location data created by the positioning system to the other component parts of the system to which it is communicatively networked is contemplated. Thus, contemplated wireless technologies include, but are not limited to, telemetry control, radio frequency communication, microwave communication, GPS and infrared short-range communication.

The systems and methods described herein comprise sensors for physical detection to detect patterns, and particularly changes to those patterns, in human or other animal respiration and/or cardiac function that are indicative of declining or altering health or indicative of upper or lower respiratory infection, disease, allergy, viral contamination or other issues of interest. As the physical detection can be sufficiently accurate to detect small changes imperceptible to a human user (and even a trained medical professional), the detection of potential illness can take place early in an infection or disease cycle and well in advance of the human identifying or suffering from symptoms. In an embodiment, the physical detection is specifically the movement of the human's chest or torso as they breathe and/or their heart beats.

This disclosure will often discuss the need to detect changes in respiration in conjunction with the determination of whether or not a user is currently infected with a particular disease and, more specifically, is capable of infecting others with that disease. It should be recognized that such determination will generally be imperfect and the system cannot definitely state that any individual, at any given time, is or is not contagious to any other specific individual as too many variables go into that determination. Instead, the purpose of these systems and methods is to provide improved gatekeeping in such detection. Specifically, the systems and methods discussed herein are designed to better determine when any individual is not contagious to most people compared to existing systems which typically only test individuals that already are at increased risk of being contagious to determine if they are.

Further, while the present disclosure will discuss the use of the systems and methods to detect transmissible infection, it should be recognized that any system which allows for more regular monitoring of human body function provides not only the ability to detect transmissible disease infection, but the ability to detect alteration of function which may be indicative of individual deterioration. Thus, while the systems and methods discussed herein provide for a number of benefits with regards to avoiding the need for broad lockdowns or gathering restrictions with regards to contagious diseases, they can also serve to provide increased data for health monitoring individually and improved health outcomes for individuals and populations.

Detection of potential disease or deterioration using the present systems and methods can occur in privacy or isolation for the patient and with the patient remote from a health care professional Detection may also be performed relatively non-invasively as the sensor is typically external to the body and may be placed external to clothing or other covers. As such, the data collection may be used in public areas to allow access to a gathering or other human activity where the presence of a disease state in one individual could be dangerous to others present. This type of detection can, thus, provide advance notice of a disease state and potentially provide sufficient time for appropriate evaluation, isolation, monitoring and/or medical intervention to inhibit disease transmission or progression and/or possibly before the illness produces irreversible complications. In the case of possible contamination to other individuals, measures can also be taken to limit the spread of disease and to notify those with potential contact to an infected individual early to attempt to halt further spread. This can result in increased effectiveness (and acceptance) of lockdowns, quarantines, and other isolating measures by better limiting them only to those who are an increased risk to others.

The systems and methods may be used for quick detection of infection or changed state over a short time frame. Alternatively, they may be used for monitoring of disease state over a long term. For example, long term data of patients with degenerative illness may be obtained to monitor the disease progression. This may be used, for example, to manage effects of the disease even if it is not medically possible to eliminate the disease or inhibit further degeneration. Further, the systems and methods discussed herein may also detect acute changes in long term illness which could indicate an immediate health concern requiring an immediate health response to prevent permanent injury or death.

In an embodiment, the systems and methods herein make use of the mechanical movement of the chest and other externally visible body structures to detect the current state of the lungs and/or heart (which are not directly visible). The sensors (101A), (101B), and (101C) may be a part of a larger monitoring system (100) for a patient (103), (105), and (107). The system may include other sensors (208), (209), or (210) where the readings are collated at a central computer (201) or similar system. The central computer may also have access to a variety of source information such as databases (203), (205), and/or (207). All communication will typically be through a network (301). The system (100) will typically be used to monitor those in a care facility such as a nursing home or hospital, but that is by no means required and the system (100) and the sensors (101) may be used at home and monitored by an individual user such as through a mobile communication device.

The sensors discussed herein may be tethered to the subject such as being placed against their chest, abdomen, or back and held in place with a strap or similar structure around their torso. They may also be simply positioned to rest on the chest, abdomen, or back utilising gravity to hold them in place or may be held in place with a hand or similar restraint as shown with patient (103). Alternatively, the sensors may be placed on a bed or chair under a user (regardless of their orientation to the bed or chair), as shown with patient (105) and (107), or may be carried by a user or otherwise held in proximity to certain points of their body, or held in place with a strap or similar structure around their torso. While location on or near the chest to obtain information on heartrate and respiration is generally preferred, it should be recognized that the sensor can be placed elsewhere on the body. For example, it may be placed on an arm to detect, for example, involuntary muscle movement within the arm or over the lower torso to detect, for example, intestinal motion, diaphragm motion, or motion of a fetus during pregnancy.

It is generally preferred that the sensors (101) be untethered from the patient (that is not connected to the patient) and placed on or in a bed (patient (105)) or mattress (115) or on or in a chair (117) (patient (107)), regardless of their orientation to the bed or chair, or may be carried by a user or otherwise held in proximity to certain points of their body. When a sensor (101) is placed on or near the body, acceleration, g-force, angular velocity and magnetic field changes to the chest area, amongst other things can be measured. External changes to the human condition (for example, that the chest rises and falls with inhalation and following exhalation) will commonly cause physical changes in the chest while the heart beats and the subject inhales and exhales.

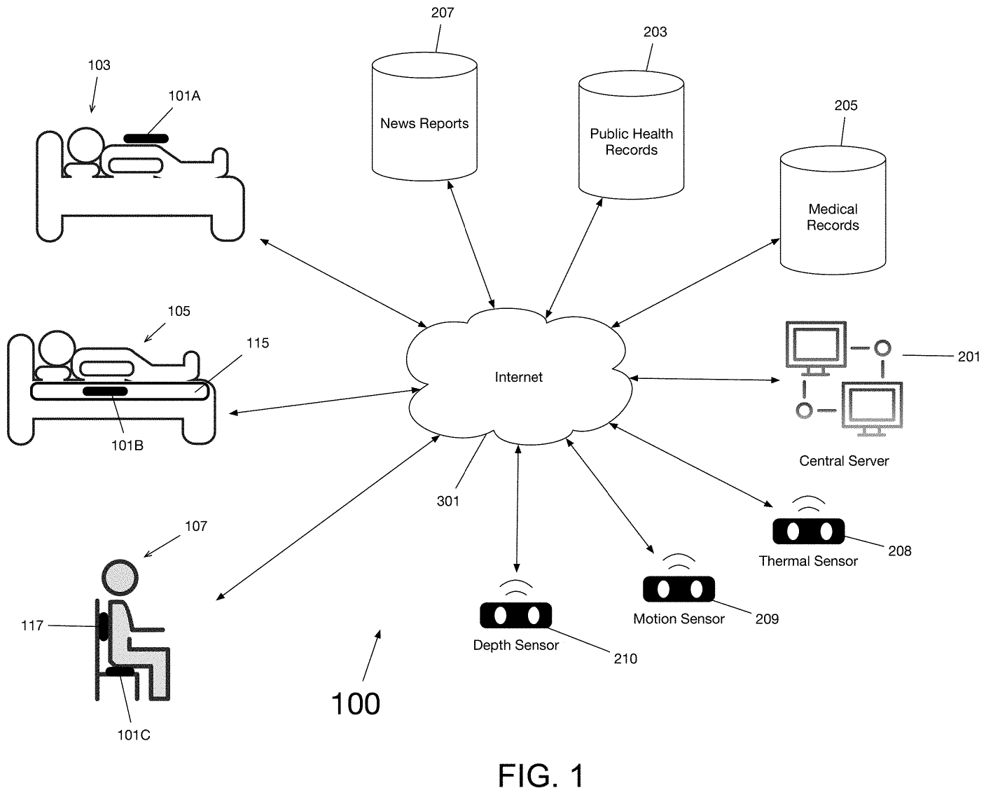

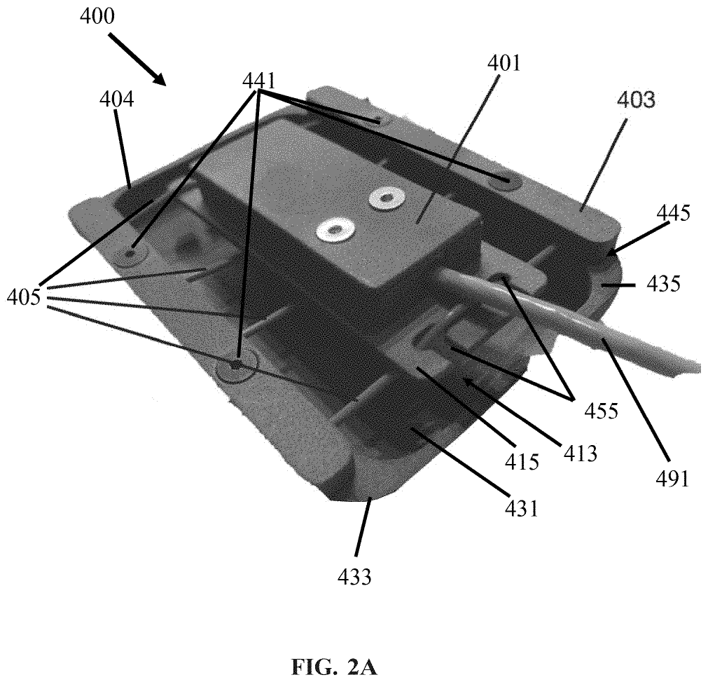

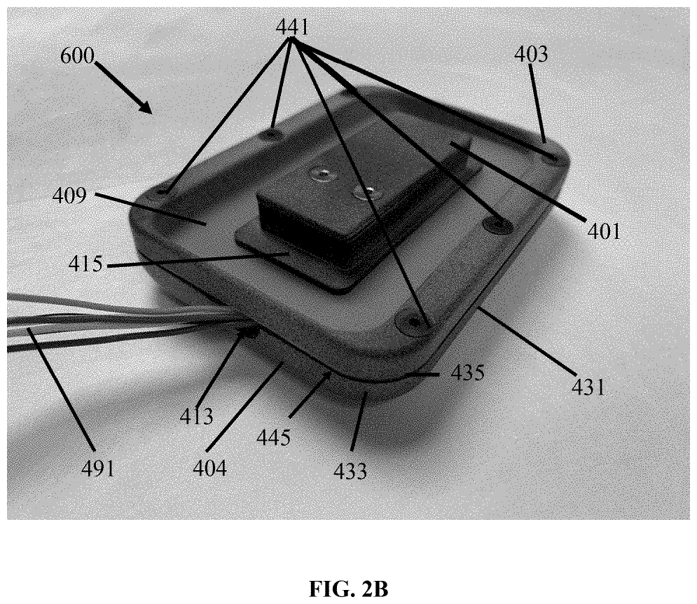

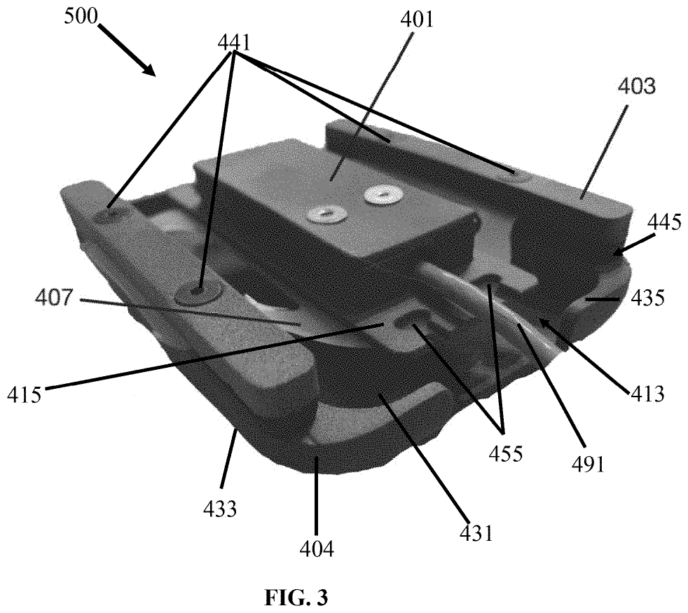

FIGS. 2A, 2B, and 3, provide for sensors specifically designed for the purpose of measuring chest, abdomen, or other body part movement, particularly of a prone or sitting patient. These are useable as sensors (101) in FIG. 1. In FIGS. 2A, 2B, and 3 an accelerometer (401) is mounted to a support (404) via a spring (405), (407), or (409). In FIG. 2A, the spring (405) simply comprises elastic, rubber, or similar bands or other supports having internal springiness. FIG. 2B is similar, but the spring (409) comprises an elastic flat surface material such as, but not limited to, silicone or rubber, instead of bands. In FIG. 3, the spring (407) comprises a piece of flat spring steel, a leaf spring, a flat spring or similar component interconnecting the accelerometer (401) and support (404). It would be understood by one of ordinary skill in the art that alternative spring structures including without limitation, coil or disk springs, could be used in alternative embodiments.

The support (404) in all of FIGS. 2A, 2B, and 3 is generally in the form of a trough having a recessed base (431) and a surrounding outer wall (433). The outer wall (433) need not be of even height throughout the entire structure and need not be continuous across its entire length. Generally, the base (431) will, however, be recessed below the upper rim (435) of the outer wall (433). The spring (405), (407), or (409) will typically be attached to the upper rim (435) and extend generally linearly across the base (431) so as to space the accelerometer (401) above the base (431) (“floating”) and create a void (413) between the base (431) and a plate (415) upon which the accelerometer (401) is mounted.

The spring (405), (407), or (409) will often be connected to the support (404) by having a portion of its structure sandwiched between the upper rim (435) and the lower surface (445) of a retaining block (403). The retaining block (403) will often include tightening connectors (441) such as, but not limited to, bolts or screws which allow it to be placed against the upper rim (435) and/or a portion of the spring (405), (407), or (409). These tightening connectors (441) may then be used to compress the lower surface (445) into the upper rim (435) and portions of the spring (405), (407), or (409). The spring (407) may also or alternatively include holes in its structure allowing it to be trapped by the tightening connector (441) itself or may be attached via other methods such as, but not limited to, adhesives, welding, co-molding, or co-formation.

The spring (405), (407), or (409) may be attached to the plate (415) in similar fashion to the support (404) by having the plate (415) comprise multiple parts also connected by tightening connectors, adhesives, or any other methods, means, or structure. However, the plate (415) will often be of simplified construction. In the depicted embodiments of FIGS. 2A and 3, the plate (415) includes a plurality of cutouts (455) through which the spring (405) may be threaded in a woven fashion so as to generally entangle the plate (415) in the spring (405). This same shape of plate (415) is used in FIG. 3, however, in this the cutouts (455) are not used as the plate is attached to the spring (407) by adhesives or similar materials. In FIG. 2B, the cutouts (455) have been eliminated. In alternative embodiments, these connection methods may both be used on the same device.

The plate (415) will generally be attached to the spring, (405), (407), or (409) in a manner that allows for the plate (415) and attached accelerometer (401) to be suspended above the void (413) in a position where the spring biasing force is such that the position is in equilibrium. Typically, the spring will be selected so that the mass of the plate (415) and accelerometer (401) is insufficient to result in deformation of the spring (405), (407), or (409) of any significant amount regardless of orientation of the sensor (400), (500), or (600), but this is by no means required. The connection mechanism can be any which allows the plate (415) to be compressed into the void (413). Typically, the plate (415) and accelerometer (401) will have a default position where the plate (415) is “floating” above the void (413) and can be compressed into the void (413) or extend out from the void (413) depending on the force placed upon it.

It should be recognized that while the sensors (400), (500), and (600) utilize springs (405), (407), and (409) which are not generally located within the void (413), this is not absolutely required. In an alternative embodiment, a coiled compression spring or similar compression spring such as, but not limited to a leaf spring or flat spring, can be attached to the base (431) and interconnected to the plate (415) as this also allows for the plate (415) to “float” above the void (413) even though the spring is within the void (413).

The void (413) allows for the plate (415) and accelerometer (401) to be compressed from its default position above the void (413) into the void (413) without movement of the support (404). By connecting the plate (415) to the wall (433) of the support (404), the compression force is distributed around the edge of the base (431). This can improve the likelihood of the plate (415) moving within the void (413) as opposed to a force on the plate (415) moving the entire sensor (400), (500), or (600). After compression, the plate (415) and accelerometer (401) will typically return to the default position and may actually extend further briefly depending on the amount to which it was compressed when any compression force is removed. This construction of sensors (400), (500), and (600) allows for the accelerometer (401) to be moved relative to the support (404) quite easily when under a compression force while at the same time providing sufficient support for the accelerometer (401) that it can obtain a signal from its movement even if it is a relatively small perturbation. This structure allows for detection of relatively small movement of a nearby object while also accurately detecting its intensity.

In operation, the sensors (400), (500), and (600) will typically operate as follows. The sensor (400), (500), or (600) will be placed in a location that will generally end up being in contact with a patient's chest or in contact with another object which would transmit motion of the patient's chest to the accelerometer (401). In alternative embodiments, other portions of the patient may alternatively or additionally be proximate the sensors (400), (500), and (600). For example, the sensor (400), (500), or (600) may be placed near a large artery or vein (such as, but not limited to, those in the neck, wrist, or upper legs) or may be placed in an area where body vibration is readily transmitted (such as, but not limited to, against large bones or against the back).

In a preferred embodiment, the sensors (400), (500), or (600) are placed in, on, or under a mattress or chair cushion so that when a user's mass is placed against them, the biasing of the spring (405), (407), or (409) is partially countered by their mass. This position allows for movement of the patient's body to perturb the accelerometer (401) relative the support (404). The support (404) will typically be placed on the side opposing the patient so that the patient will be closer to the accelerometer (401) than the support (404). Thus, in a mattress arrangement, the support will typically be “down” and the accelerometer “up”. This is by no means required, however.

In an alternative arrangement, the sensor (400), (500), or (600) may be positioned so as to be directly attached to the patient. For example, the sensor (400), (500), or (600) may be placed on the patient's torso and attached to them via a strap. In such an arrangement, the support (404) will again typically be furthest from the patient so the accelerometer (401) would be closest to them and often in contact with their skin or clothes.

Regardless of the particular mounting, it should be apparent that small changes in the pressure being applied to the sensor (400), (500), or (600) from the patient's chest moving will generally be readily detected as movement of the accelerometer (401) relative to the support (404). Further, large scale movements of the user's body will often result in movement of both the accelerometer (401) and the support (404). This can allow for differentiation in signals. Specifically, smaller movements that will typically generate less force such as chest movement due to the user's breathing or heartrate will serve to perturb the accelerometer (401) relative to the support (404) by a relatively small amount while a large movement (such as the user's rolling over while on the sensor (400), (500), or (600) will serve to move both the accelerometer (401) and the support (404). This movement may be detectable by the large size of the signal received by the accelerometer (401) which would indicate that the accelerometer (401) moved further than expected due to movement of the support (404). The accelerometer (401) may not also return to the same position it started showing a greater movement in one direction. These can indicate that the movement is caused by a gross motor movement as opposed to a desired perturbation such as inhalation or heartbeat.

Once positioned, the sensor (400), (500), or (600) can serve to provide indications of accelerometer (401) motion to an analysis system. This will typically be a computer and may receive the signals from the sensor (400), (500), or (600) via a cable (491) or via a wireless transmission if the accelerometer (401) is so equipped. These signals are typically indicative of the movement of a patient's chest or other body structure and can be used to monitor breathing, heartbeat, or other factors of the patient.

The combination of waveforms from these sensors can be processed by algorithms to identify heartbeats and estimate respiration or heart operation features or patterns. Certain combinations of features (for example, specific waveforms of inhalation, timing of heartbeat, or shape of waveform caused by heartbeat or inhalation) are then correlated to early signs of potential infection, respiratory issues or other illness. This correlation can be carried out at a macro scale level where waveforms from one specific user are compared to waveforms gathered from a large group of other users both with and without respiratory conditions of interest. Alternatively, or additionally, changes in waveforms patterns over time for each person individually (comparing current waveforms from that individual against prior waveforms from that individual) may also be correlated to signs of potential infection, respiratory issues, or other illness.

Monitoring of changes in the patterns over time typically allows for more accurate identification of potential illness (less false alarms), earlier in time, allows for more conditions to be detected and also allows for the system to adjust on a personal level for each individual. Adjusting for each individual is generally preferred as each person's “regular” condition is different. That is, each person's physiology typically results in them generating different waveforms. However, both the features indicating illness and the deteriorating health patterns can be predetermined by experts in the field (such as pulmonologists or cardiologists) or learned by the system over time (such as through the use of a neural net or other “artificial intelligence” technology) for each person.

This may be accomplished, for example, by establishing a baseline set of features and parameters and monitoring for changes. As an example, coughing, shortness of breath (gasping breaths), number of respirations per minute, depth of breath, completeness of breath, or timing or shape of heartbeat could all be estimated by the waveforms of the aforementioned sensory equipment and the values or changes of values over time correlated to particular irregularities due to illness. As another example, signs of cardiovascular compensation for incomplete blood oxygenation can be identified by evaluating simultaneously changes in heart and respiratory rates.

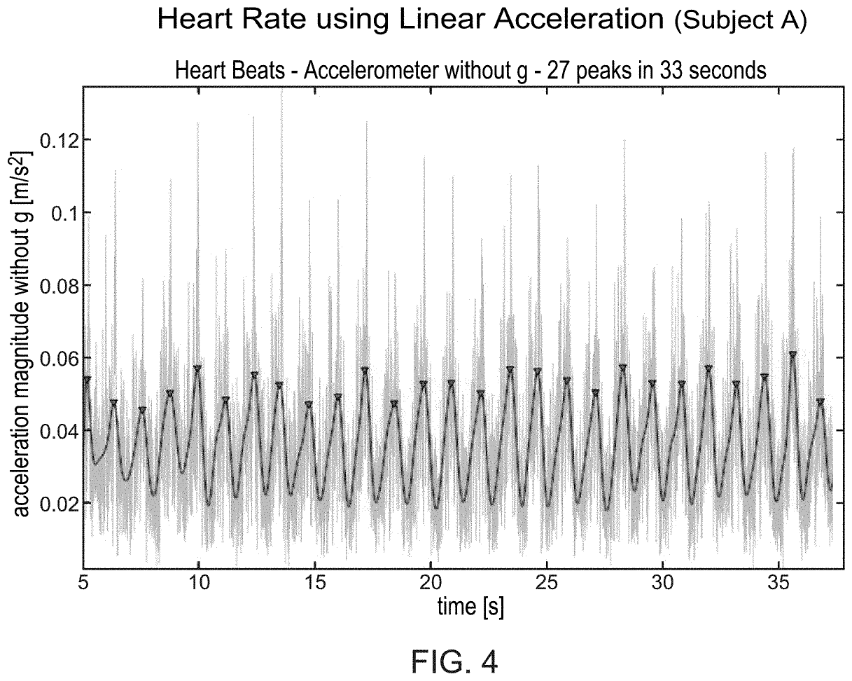

FIGS. 4 through 8 provide for various graphs of waveforms which are indicative of physical characteristics of particular patients. FIG. 4 provides for an indication of the detection of heart rate via linear acceleration as detected by a sensor such as sensor (400), (500), or (600). As should be apparent, individual beats are easily detected and the heartrate can be readily determined by simply reviewing the beats over time.

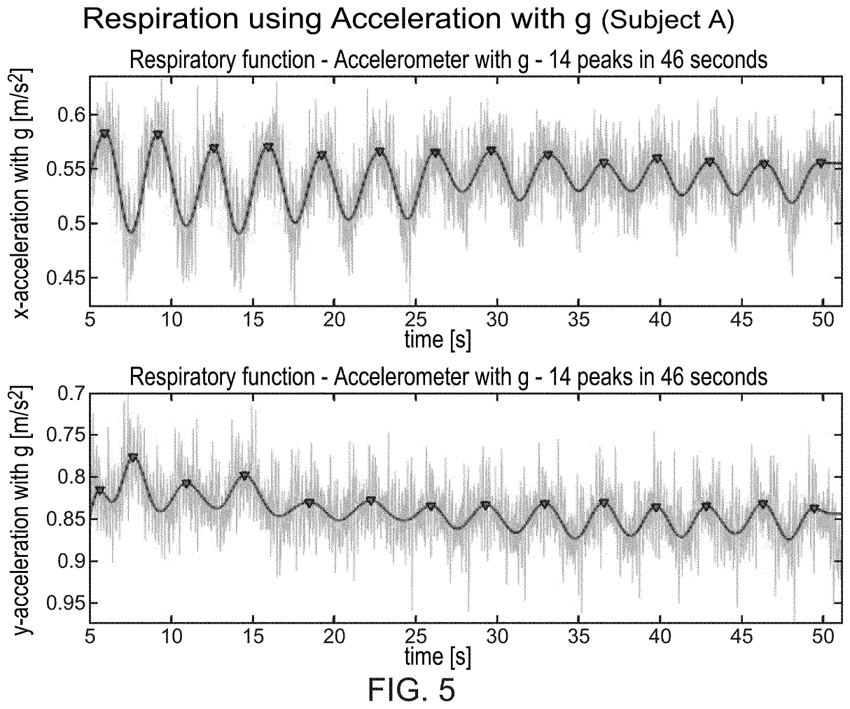

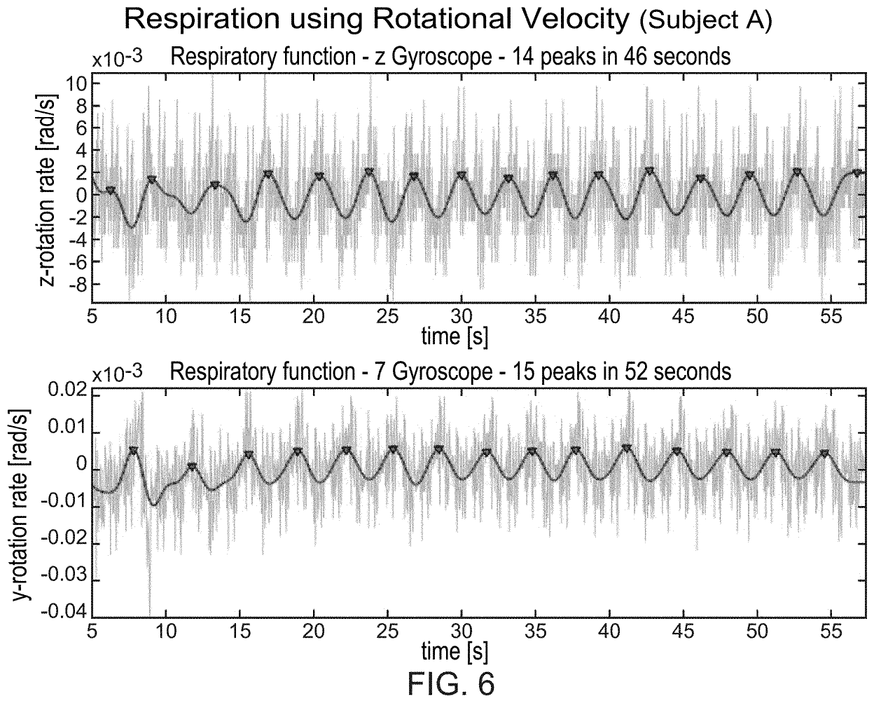

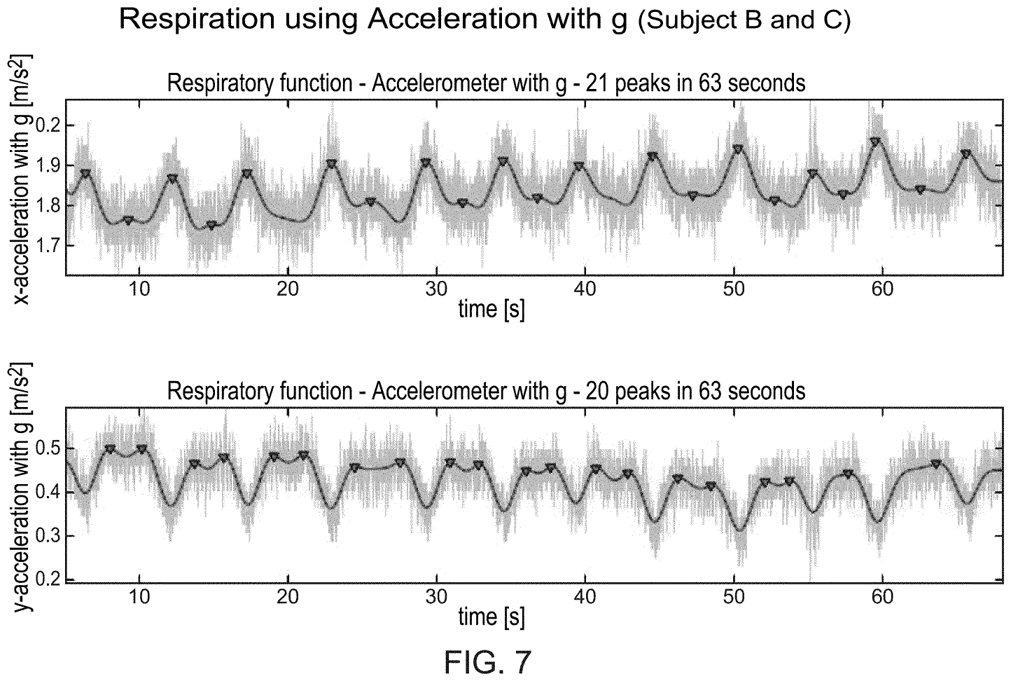

FIGS. 5 through 7 provide for various different graphs showing patterns of respiration. These are generated via various different forms of sensing of acceleration from sensors such as sensors (400), (500), or (600). In FIG. 5, acceleration with g is used. FIG. 6 uses rotational acceleration on the same patient as FIG. 5. FIG. 7 utilizes acceleration with g for two different patients than FIG. 5. As should be apparent, the graphs (which may be referred to as “Respirographs”) in FIG. 7 show differences from each other with the top graph showing longer troughs while the lower graph shows longer more flat-topped peaks. This is compared to FIG. 5 which shows a more regular sinusoidal type wave. While the various graphs are from different individuals, they clearly show different ways of breathing. Some of the differences may be pathological (for example, subject B suffers from asthma) or may simply comprise differences from individuals. The software is able to determine differences both within an individual's pattern over time and between individuals with or without known conditions (for example if they do or do not have asthma) and can alert accordingly for detected changes or the potential presence of chronic conditions.

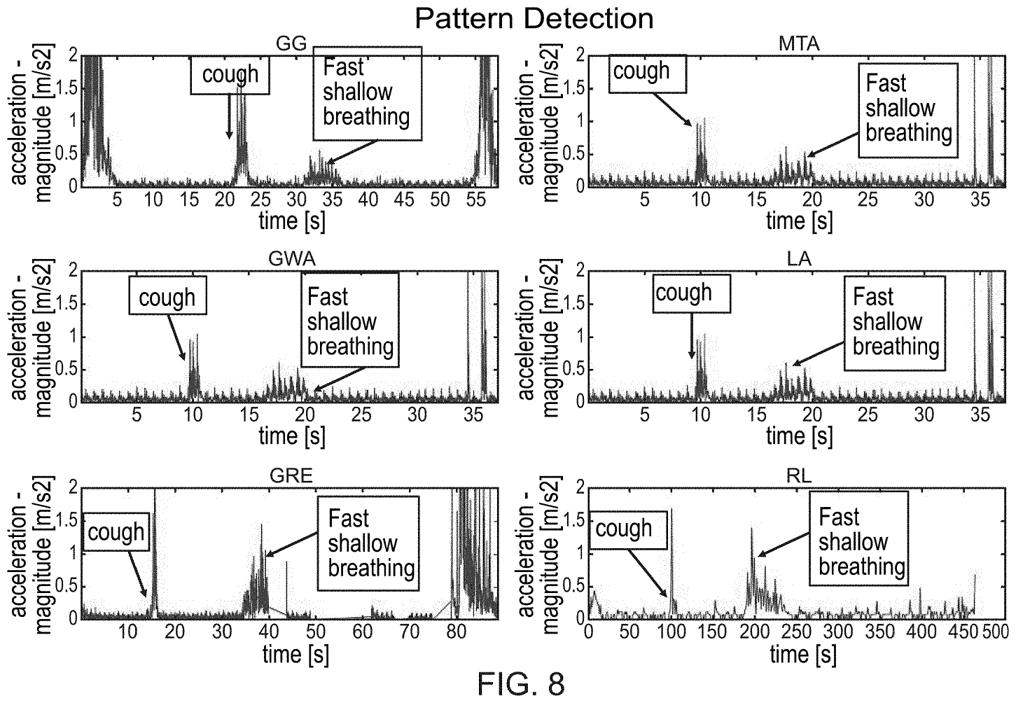

FIG. 8 shows that particular patterns in breathing can be detected across patients. Specifically, the 6 graphs of FIG. 8 show six different patients using sensors such as sensors (400), (500), or (600). The patients each cough and have that followed by a period of fast shallow breathing. These elements are each readily detectible on all six graphs. Further, the cough is also clearly differentiable from the shallow breathing. As would be expected, fast shallow breathing results in dramatically increased acceleration while the cough produces a large acceleration from the force produced by the muscles of the chest to cough. These structures and corresponding actions are easily identified, even though each patient has different specific graphs as can be seen.

While the above FIGS. show that individual differences in breathing can be readily detected (FIGS. 5 through 7) it also shows that common actions such as coughing or changed breathing (FIG. 8) can also be readily detected. This dual detection can be used to monitor an individual and/or population to detect both acute concerns as well as overall changes.

FIG. 1 illustrates an embodiment of a system for collating respiration information from a number of users. Human users (103), (105), and (107) would be provided with sensors (400), (500), or (600) in various locations. In one example, a user (103) could have a sensor (101A) placed on them while lying as illustrated by user (103). The sensor (101A) could sense strength of heartbeat and pace of respiration or other factors of the user to determine if the location is as desired for measurement. Should the measurement window be sufficient, other aspects of breathing such as a cough, sneeze, or hiccup could be detected. It should be recognized that a patient that has respiratory distress is far more likely to cough or sneeze during the measurement window. Further, patients that are having difficulty breathing are more likely to have their breathing become quicker.

Alternatively, the sensor (101B) may be placed on, under or within the mattress (115) of a patient (105) in a nursing home who is unable to leave bed without assistance. This device (101B) may operate according to a set time schedule or even constantly instead of when activated by the user or under specific circumstances. A still further sensor (101C) could be placed in a chair (117) in the room of a patient (107) in a hospital isolation ward and simply activated by pressure when they sat down. In the depicted embodiment sensor (101C) is actually two linked devices as shown to provide different monitoring locations.

The user (103) may be instructed to breathe normally or to carry out a specific series of breathing exercises such as specifically taking the deepest breaths possible or to breathe and hold their breath for a period of time. They could also be asked specifically to cough or to move in a particular way. The users (105) and (107) may be similarly instructed or may be passively monitored. Regardless, the sensors (101A), (101B), or (101C) would then record the data related to the chest and/or abdominal movement of the user (103), (105), or (107) during these actions.

This data could then be transferred using a transmitter or similar device to a server (201) via the Internet (301) or another network. The data may be packaged with other data from the sensors (101) such as time or location information. A user (103) or a caregiver for a user (105) or (107) may also be specifically requested to enter additional information in the transmission such as a current health state, if they had difficulty performing any of the breathing exercises, or if they believe they may have been exposed to a certain illness since the last time they utilized the sensor. This user-supplied data can also be combined with the collected motion data.

Data from the sensors (101) may also be combined with data from other monitoring systems such as, but not limited to, systems in the room for monitoring the patient for other conditions. For example, thermal sensors (208) may be used to make sure that the data obtained by the mobile device (101) appears to be from a human patient in the room and not a different signal. This is similar to how such thermal signals may be used to detect a human user for fall detection as discussed in U.S. Pat. No. 10,453,202 the entire disclosure of which is herein incorporated by reference. Thermal sensors (208) may also be used to determine if a patient is feverish in an embodiment such as is described in United States Patent Application Publications 2008/0154138, 2007/0153871, and 2016/0150976, the entire disclosure of all of which is herein incorporated by reference.

Similarly, monitoring systems such as motion sensor (209) and depth sensor (210) which may be in the room for fall detection such as is described in U.S. Pat. Nos. 8,890,937; 9,408,561; 9,597,016; 10,080,513; 10,188,295; and 10,206,630, the entire disclosures of all of which are herein incorporated by reference, may also supply data on the patient which may be combined with the data from the sensor (101). Any and all data may be sent to the server (201) encrypted and/or anonymized to protect privacy of the user (103), (105) or (107).

The central server (201) may receive the data from a plurality of sensors (101A), (101B), and (101C) and may collate and analyze the data from this plurality for patterns and correlations. If certain patterns are detected, this may be combined with general information available from databases (203), (205), and/or (207) to which the server (201) has access. For example, if the server (201) received information from sensors (101) in location A which appeared anomalous, the server (201) may look to public health databases (203) or news databases (207) to determine if there may be an outbreak of a particular illness (such as, but not limited to, flu or SARS) reported in location A.

The server (201) may also obtain specific health related information related to the patterns. For example, information collected by other monitors (such as, for example an electrocardiogram, blood oxygenation reading, diagnosis, and eventual outcome) could be obtained from (typically anonymized) medical records (205) of a patient that was hospitalized for a certain condition after showing similar pattern changes in breathing.

If certain elements of the data received from the sensor (101) of any user are determined to potentially be indicative of a certain illness or condition, the system (100) may notify the user of the mobile device (101) that such has been found and suggest they consult a health professional. The data may additionally or alternatively be passed to a health professional or caregiver that the user may have already indicated as authorized to receive their information. This person could then evaluate the data and conclusion and potentially contact the user directly, or institute care or monitoring, if they deemed such actions relevant.

While the invention has been disclosed in conjunction with a description of certain embodiments, including those that are currently believed to be useful embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

It will further be understood that any of the ranges, values, properties, or characteristics given for any single component of the present disclosure can be used interchangeably with any ranges, values, properties, or characteristics given for any of the other components of the disclosure, where compatible, to form an embodiment having defined values for each of the components, as given herein throughout. Further, ranges provided for a genus or a category can also be applied to species within the genus or members of the category unless otherwise noted.

The qualifier “generally,” and similar qualifiers as used in the present case, would be understood by one of ordinary skill in the art to accommodate recognizable attempts to conform a device to the qualified term, which may nevertheless fall short of doing so. This is because terms such as “spherical” are purely geometric constructs and no real-world component or relationship is truly “spherical” in the geometric sense. Variations from geometric and mathematical descriptions are unavoidable due to, among other things, manufacturing tolerances resulting in shape variations, defects and imperfections, non-uniform thermal expansion, and natural wear. Moreover, there exists for every object a level of magnification at which geometric and mathematical descriptors fail due to the nature of matter. One of ordinary skill would thus understand the term “generally” and relationships contemplated herein regardless of the inclusion of such qualifiers to include a range of variations from the literal geometric meaning of the term in view of these and other considerations.

1. A sensor for detecting cardiac function or breathing of an animal, the sensor comprising:
a support formed of a base and an outer wall;
an accelerometer; and
a spring, said spring at equilibrium suspending said accelerometer above said base;
wherein when said spring allows for said accelerometer to be moved relative to said base.

2. The sensor of claim 1 wherein said animal is a human.

3. The sensor of claim 1 wherein said spring comprises elastic bands.

4. The sensor of claim 1 wherein said spring comprises an elastic flat surface material.

5. The sensor of claim 1 wherein said spring comprises flat spring steel.

6. The sensor of claim 1 wherein said spring comprises a leaf spring.

7. The sensor of claim 1 wherein said spring comprises a coil spring.

8. The sensor of claim 1 wherein said spring comprises a disk spring.

9. The sensor of claim 1 wherein said spring is attached to said outer wall.

10. The sensor of claim 1 wherein there is a void between said spring and said base which said accelerometer can move into.

11. A system for detecting cardiac function or breathing of an animal, the system comprising:
a sensor comprising:
a support formed of a base and an outer wall;
an accelerometer; and
a spring, said spring at equilibrium suspending said accelerometer above said base;
wherein when said spring allows for said accelerometer to be moved relative to said base; and
a support structure, mounting said sensor in a fashion that movement of a human torso through respiration or cardiac function causes said accelerometer to move relative to said base.

12. The system of claim 11 wherein said support structure comprises a bed.

13. The system of claim 11 wherein said support structure comprises a chair.

14. The system of claim 11 wherein said support structure comprises said human torso.

15. The system of claim 11 further comprising a central computer for receiving signals from said accelerometer via a network.

16. The system of claim 15 further comprising a thermal sensor.

17. The system of claim 15 further comprising a motion sensor.

18. The system of claim 15 further comprising a depth sensor.

19. The system of claim 15 wherein said central computer can compare said received signals against other signals.

20. The system of claim 20 wherein said other signals comprise prior received signals.
